(19) United States
(12) Patent Application Publication
(10) Pub. No.: US 2022/0370189 A1
(43) Pub. Date: Nov. 24, 2022

(54) ARTIFICIAL ESOPHAGEAL STRUCTURE HAVING MULTI-LAYER STRUCTURE USING THREE-DIMENSIONAL BIO-PRINTING, AND MANUFACTURING DEVICE AND MANUFACTURING METHOD THEREFOR

(71) Applicants: WONKWANG UNIVERSITY CENTER FOR INDUSTRY-ACADEMY COOPERATION, Jeotiabuk-do (KR); POSTECH RESEARCH AND BUSINESS DEVELOPMENT FOUNDATION, Gyeongsangbuk-do (KR); THE CATHOLIC UNIVERSITY OF KOREA INDUSTRY- ACADEMI COOPERATION FOUNDATION, Seoul (KR)

(72) Inventors: Seung Jae LEE, Jeollabuk-do (KR); Jin Ah JANG, Gyeonggi-do (KR); Young Sam CHO, Daejeon (KR); Kyoung Duck SEO, Jeollabuk-do (KR); Hun Jin JEONG, Seoul (KR); Hyo Ryung NAM, Gyeongsangbuk-do (KR); Ji Hyun KIM, Seoul (KR); Jae Hee CHUNG, Seoul (KR); Yeong Gwon JO, Seoul (KR); Dong Woo CHO, Gyeongsangbuk-do (KR); Dong Heon HA, Gyeongsangbuk-do (KR)

(21) Appl. No.: 17/764,055

(22) PCT Filed: Sep. 3, 2020
(86) PCT No.: PCT/KR2020/011880

§ 371 Date: Mar. 25, 2022

(30) Foreign Application Priority Data
Sep. 26, 2019 (KR) 10-2019-0118951

(51) Int. Cl. A61F 2/04; A61L 27/56; A61L 27/38

(52) U.S. Cl. CPC A61F 2/04; A61L 27/56; A61L 27/3882; A61F 2002/044; A61F 2240/002; A61F 2002/0081

(57) ABSTRACT

Disclosed are an artificial esophageal structure having a multi-layer structure using three-dimensional bio-printing, and a manufacturing device and manufacturing method therefor. The artificial esophageal structure having a multi-layer structure according to one embodiment of the present invention comprises: a first layer in the shape of a hollow column and having a circular cross section; a second layer which is disposed inside the first layer and which is a column structure that simulates the mucosal layer of the esophagus; and an interlayer support part which is disposed between the first layer and the second layer and which maintains a gap between the layers, wherein the first layer and second layer each comprise: a plurality of column parts disposed at predetermined intervals; and a plurality of strands formed between the plurality of column parts by a dragging technique, and may have a porous structure due to pores between the plurality of strands.

### TECHNICAL FIELD

The present invention relates to an artificial esophageal scaffold having a multi-layered construct using three-dimensional bioprinting, a manufacturing apparatus, and a manufacturing method.

### RELATED ART

The esophagus is a digestive organ between the pharynx and the stomach, and serves to move food into the stomach through contraction and relaxation. Diseases such as esophageal cancer, malignancy, and esophageal varix may occur in the esophagus. Among them, esophageal cancer has few symptoms in the early stages, making it difficult to recognize the onset, and unlike other organs, the esophagus has a thin tissue and no outer membrane, so esophageal cancer metastasizes quickly to nearby organs, and is a dangerous cancer that ranks sixth in the world's cancer mortality rate.

In this case, surgery is performed to excise and remove the esophagus at the affected area, but unlike bone and cartilage tissue, autologous tissue cannot be transplanted, so esophageal-gastrointestinal anastomosis and esophageal-colorectal-gastrointestinal anastomosis are performed. However, since this method has a high complication rate of 30-40% or more, it is essential to develop an artificial esophagus that can reconstruct the lesioned esophagus.

Currently, there is no artificial esophageal scaffold applied in clinical practice, and the esophageal scaffold being researched and developed has a limitation in that it cannot mimic the multi-layered construct of the esophagus (mucosa, submucosa, and muscle layer) due to technical limitations.

### SUMMARY

### Technical Objectives

The present invention is to provide an artificial esophageal scaffold most similar to the actual structure and mechanical/chemical properties of the esophagus, and an apparatus and method for manufacturing the same, by manufacturing a porous tubular scaffold having a free multi-layered construct using a biocompatible/biodegradable polymer through three-dimensional bioprinting technology and mimicking the structure and shape of the esophagus and the cell layer by printing a bio-ink derived from a natural polymer containing cells of each layer of the esophagus therein.

The present invention is to provide an apparatus and a method for manufacturing an artificial esophagus scaffold, which is not restricted by a size of rotating mandrel and enables manufacturing a tubular scaffold having a multi-layered construct in aid of a dragging technique.

Other objectives of the present invention will become clearer through embodiments described below.

### Technical Solution

According to one aspect of the present invention, there is provided an artificial esophageal scaffold of a multi-layered construct, including a first layer in a hollow columnar shape with a circular cross section, a second layer being disposed inside the first layer and in a columnar construct mimicking a mucosa of an esophagus and an interlayer support being disposed between the first layer and the second layer and configured for maintaining the interlayer spacing, wherein each of the first layer and the second layer comprises a plurality of columns disposed at regular intervals, and a plurality of strands formed by a dragging technique between the plurality of columns, wherein it has a porous construct by pores between the plurality of strands.

The columnar construct may have a columnar shape having a star-shaped cross-section, and the columns may be disposed at a ridge and a valley of the star-shaped cross-section in the second layer.

An auxiliary column may be further disposed between the ridge and the valley.

The first layer may form an outer wrinkled construct by repeating an increase and a decrease of a diameter according to a height of a sliced horizontal layer.

A bioink may be printed in a space between the first layer and the second layer.

Both ends in a lengthwise direction of the artificial esophageal scaffold may be manufactured in a structure without pores by a plotting technique.

A size of pore may be adjusted by a distance between the columns.

The columns may be formed by a four-point injection plotting technique and are related to a flexibility of the construct.

According to another aspect of the present invention, there is provided a method of manufacturing an artificial esophageal scaffold of a multi-layered construct, including separately designing, by a controller in an apparatus of manufacturing the artificial esophageal scaffold, based on a basic design shape for the artificial esophageal scaffold of the multi-layered construct having a first layer in a columnar shape with a circular cross section and a second layer being disposed inside the first layer and in a columnar construct mimicking a mucosa of an esophagus, each layer of the artificial esophageal scaffold into a plurality of columns to be formed by a plotting technique and a strand between the columns to be formed by a dragging technique, generating G-code to which the dragging technique applied from the separately designed construct and manufacturing the columns and the strand according to the G-code, wherein when the strand is manufactured, a fan of the apparatus of manufacturing the artificial esophageal scaffold is operated to control a degree of stretch of the strand by discharging through the injection nozzle in a state in which forced convection is generated.

In the separately designing, a size of pore may be adjusted by setting pore sizes of each layer and by adjusting a distance between the columns by setting column arrangement at each layer.

The columnar construct may have a columnar shape having a star-shaped cross-section, and in the separately designing, the columns may be disposed at a ridge and a valley of the star-shaped cross-section in the second layer.

In the separately designing, the first layer may form an outer wrinkled construct by repeating an increase and a decrease of a diameter according to a height of a sliced horizontal layer.

A printing a bioink in a space between the first layer and the second layer may be further included.

Other aspects, features, and advantages other than those described above will be apparent from the following drawings, claims, and detailed descriptions of the invention.

### Effects

It is advantageous to provide an artificial esophageal scaffold most similar to the actual structure and mechanical/chemical properties of the esophagus, and an apparatus and method for manufacturing the same, by manufacturing a porous tubular scaffold having a free multi-layered construct using a biocompatible/biodegradable polymer through three-dimensional bioprinting technology and mimicking the structure and shape of the esophagus and the cell layer by printing a bio-ink derived from a natural polymer containing cells of each layer of the esophagus therein.

It is also advantageous to provide an apparatus and a method for manufacturing an artificial esophagus scaffold, which is not restricted by the rotating mandrel's size and enables manufacturing a tubular scaffold having a multi-layered construct in aid of a dragging technique.

### BRIEF DESCRIPTION OF ACCOMPANYING DRAWINGS

FIG. 1 illustrates apparatus for manufacturing conventional tubular structures;

FIG. 2 is a flowchart of method of manufacturing an artificial esophageal scaffold having a multi-layered structure by using a three-dimensional bioprinting according to one embodiment of the present invention;

FIG. 3 illustrates a cross-sectional view of the artificial esophageal scaffold manufactured by the method according to one embodiment of the present invention;

FIG. 4 and FIG. 5 are pictures of the artificial esophageal scaffold having three-layered structure that is actually manufactured;

FIG. 6 is a picture of the artificial esophageal scaffold having two-layered structure that is actually manufactured;

FIG. 7 schematically illustrates an apparatus of manufacturing the artificial esophageal scaffold according to one embodiment of the present invention;

FIG. 8 illustrates a process of manufacturing a conventional scaffold;

FIG. 9 illustrates a process of manufacturing the artificial esophageal scaffold to which a dragging technique according to one embodiment of the present invention is applied;

FIG. 10 illustrates the artificial esophageal scaffold manufactured by the dragging technique according to one embodiment of the present invention.

FIG. 11 is a detailed flowchart of the dragging technique design application process FIG. 12 exemplarily illustrates the dragging technique design application process;

FIG. 13 exemplarily illustrates a G-code;

FIG. 14 is a detailed flowchart of a printing process to which the dragging technique is applied;

FIG. 15 is a conceptual diagram of the apparatus of manufacturing the artificial esophageal scaffold for performing a printing process to which the dragging technique is applied;

FIG. 16 is a picture of the manufactured scaffold;

FIG. 17 is a picture of a strand;

FIG. 18 illustrates the design parameters for forming the inner wrinkles of the artificial esophageal scaffold;

FIG. 19 illustrates the design parameters for forming the outer wrinkles of the artificial esophageal scaffold;

FIG. 20 is a schematic diagram of the artificial esophageal scaffold manufacturing procedure;

FIG. 21 illustrates the types of bioink included in the artificial esophageal scaffold; and

FIG. 22 shows the appearance and flexibility test of the artificial esophageal scaffold manufactured by the method of manufacturing the artificial esophageal scaffold according to one embodiment of the present invention.

### EMBODIMENTS

The invention can be modified in various forms and specific embodiments will be described and shown below. However, the embodiments are not intended to limit the invention, but it should be understood that the invention includes all the modifications, equivalents, and replacements belonging to the concept and the technical scope of the invention.

If it is mentioned that an element is “connected to” or “coupled to” another element, it should be understood that still another element may be interposed therebetween, as well as that the element may be connected or coupled directly to another element. On the contrary, if it is mentioned that an element is “connected directly to” or “coupled directly to” another element, it should be understood that still another element is not interposed therebetween.

Terms such as first, second, etc., may be used to refer to various elements, but, these element should not be limited due to these terms. These terms will be used to distinguish one element from another element.

The terms used in the following description are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

Elements of an embodiment described below with reference to the accompanying drawings are not limited to the corresponding embodiment, may be included in another embodiment without departing from the technical spirit of the invention. Although particular description is not made, plural embodiments may be embodied as one embodiment.

In describing the invention with reference to the accompanying drawings, like elements are referenced by like reference numerals or signs regardless of the drawing numbers and description thereof is not repeated. If it is determined that detailed description of known techniques involved in the invention makes the gist of the invention obscure, the detailed description thereof will not be made.

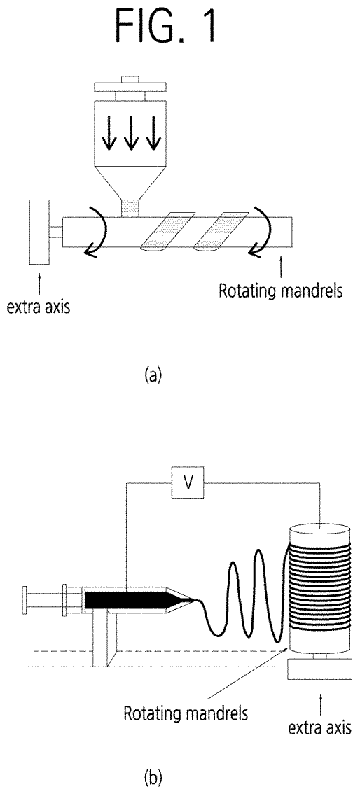

FIG. 1 illustrates apparatus for manufacturing conventional tubular structures.

Conventionally, in order to manufacture the tubular structures such as an artificial esophagus, a four-axial printing in (a) of FIG. 1 or a three-dimensional printing technology such as electro-spinning in (b) of FIG. 1 is being used.

In manufacturing the tubular structure, a rotating mandrel is required, and an extra rotating axis is required. And since the diameter of the tubular structure to be manufactured is constrained by the size of the rotating mandrel (for example, an outer diameter), the size and shape of the tubular structure manufactured according to the shape of the rotating mandrel is limited. In addition, it is disadvantageous that a free-form tubular scaffold cannot be manufactured.

Therefore, it is impossible to manufacture a tubular structure having a wrinkled, multi-layered construct like the esophagus, and to perform the three-dimensional printing with bioink derived from natural polymers with significantly lower viscosity compared to synthetic polymers such as PTFE-gore text, PET, and so on.

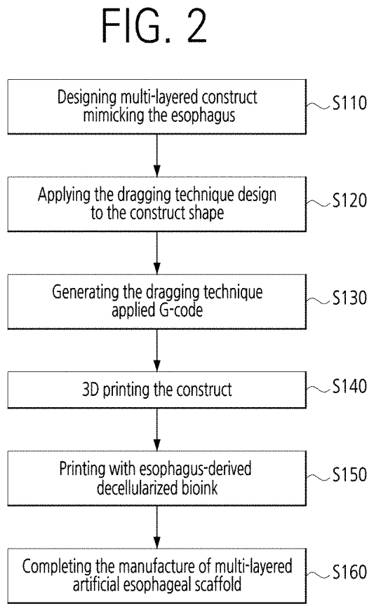

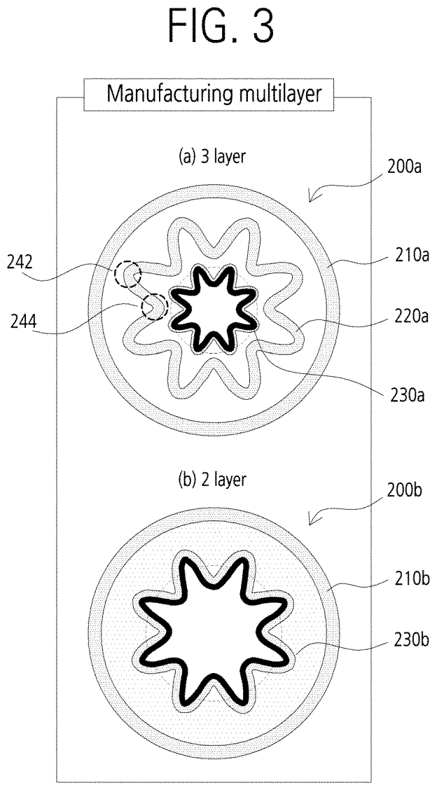

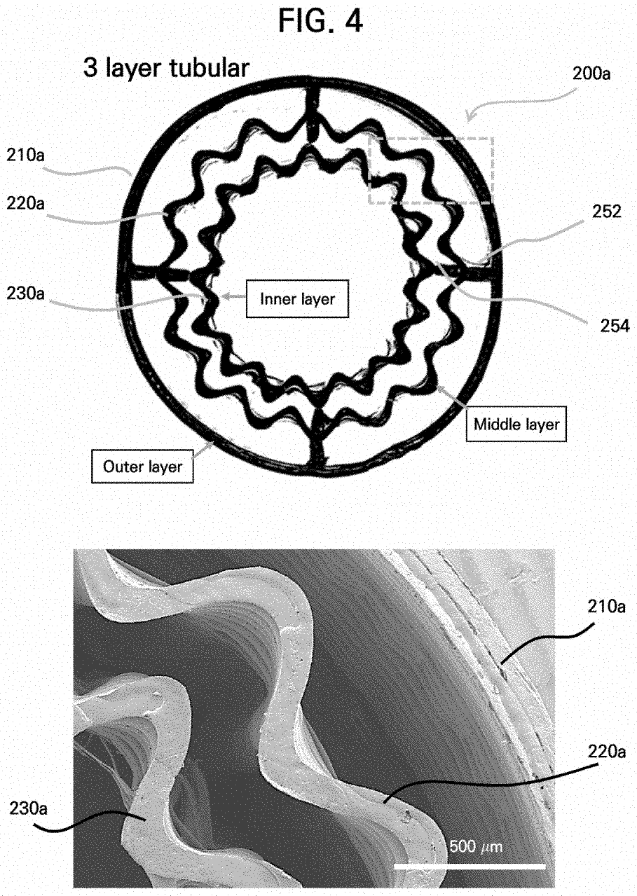

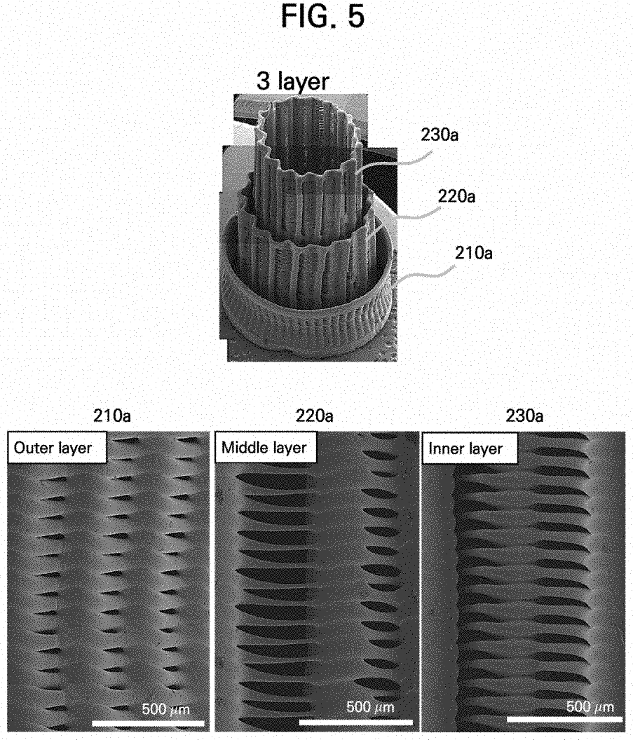

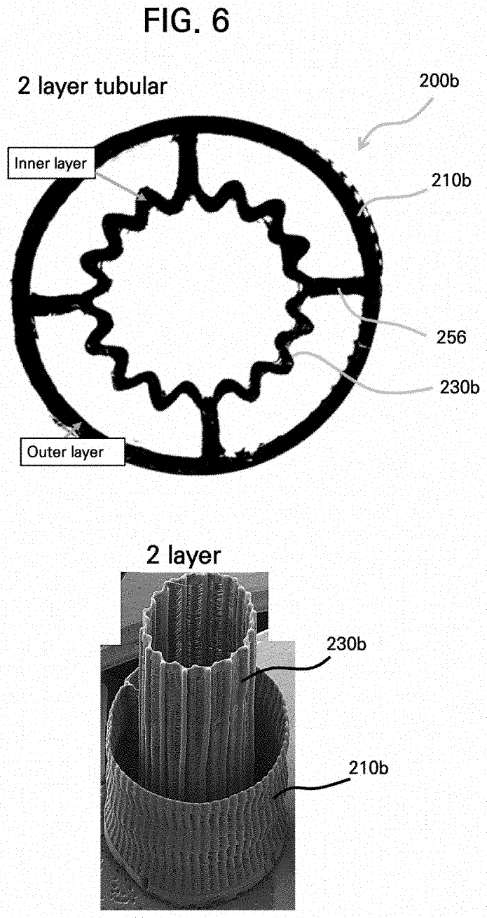

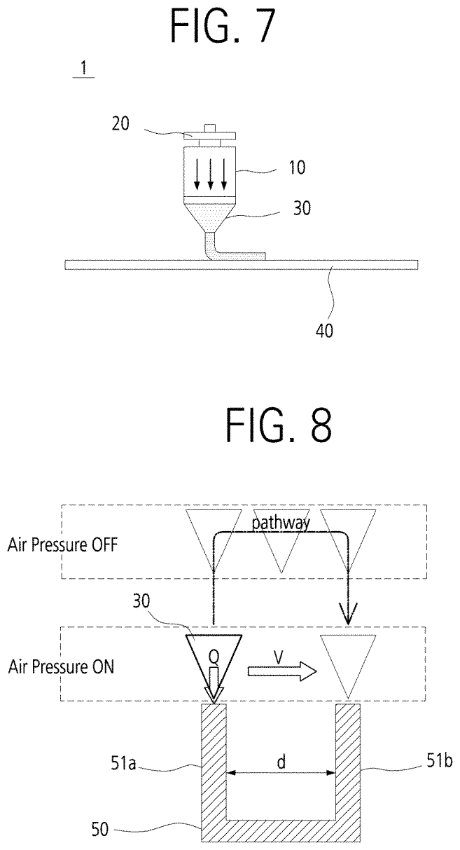

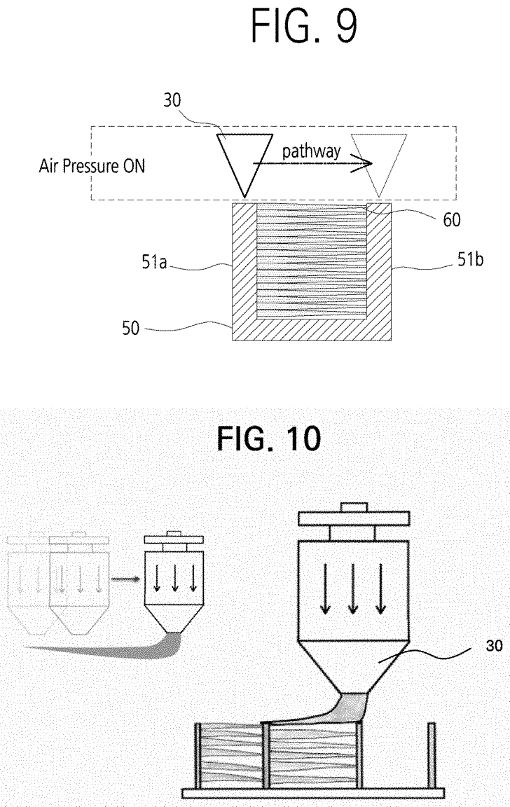

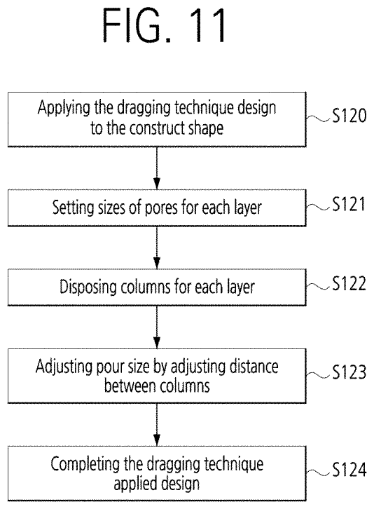

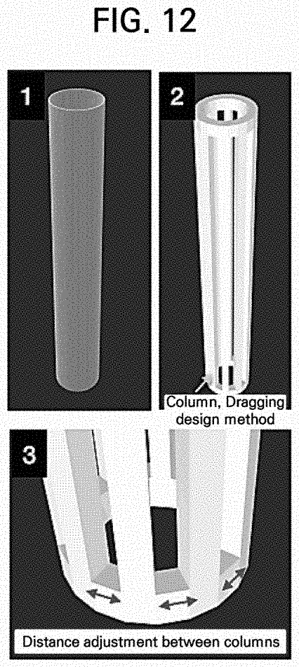

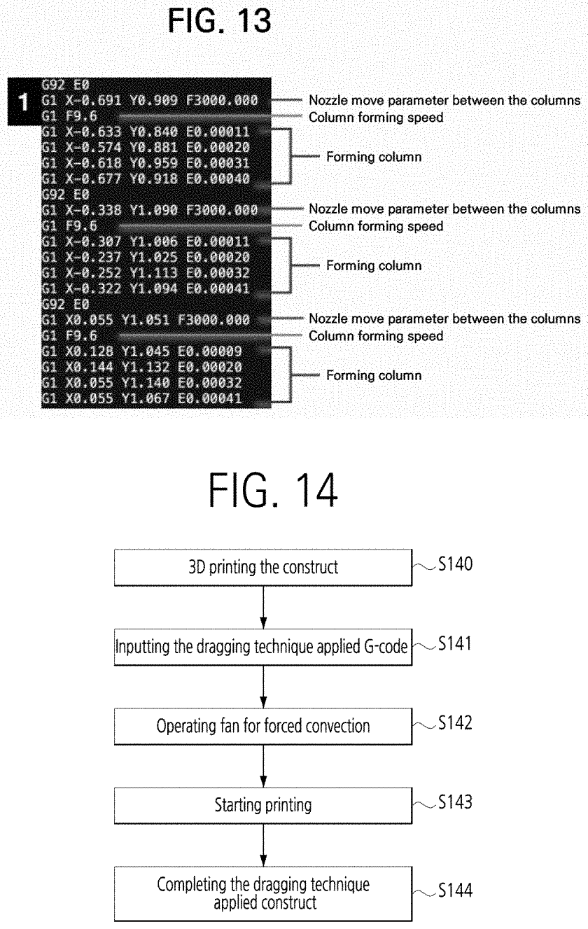

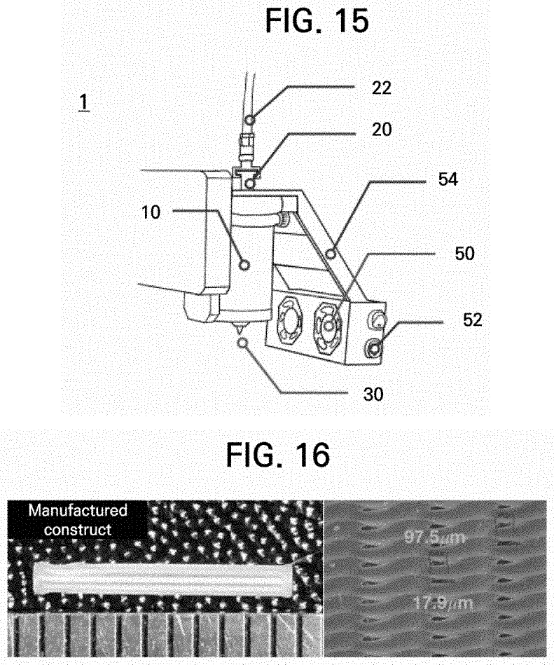

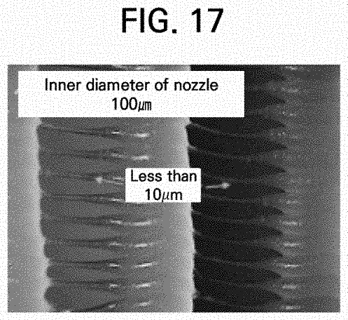

FIG. 2 is a flowchart of method of manufacturing an artificial esophageal scaffold having a multi-layered structure by using a three-dimensional bioprinting according to one embodiment of the present invention, FIG. 3 illustrates a cross-sectional view of the artificial esophageal scaffold manufactured by the method according to one embodiment of the present invention, FIG. 4 and FIG. 5 are pictures of the artificial esophageal scaffold having three-layered structure that is actually manufactured, FIG. 6 is a picture of the artificial esophageal scaffold having two-layered structure that is actually manufactured. FIG. 7 schematically illustrates an apparatus of manufacturing the artificial esophageal scaffold according to one embodiment of the present invention, FIG. 8 illustrates a process of manufacturing a conventional scaffold, FIG. 9 illustrates a process of manufacturing the artificial esophageal scaffold to which a dragging technique according to one embodiment of the present invention is applied, and FIG. 10 illustrates the artificial esophageal scaffold manufactured by the dragging technique according to one embodiment of the present invention. FIG. 11 is a detailed flowchart of the dragging technique design application process, FIG. 12 exemplarily illustrates the dragging technique design application process, FIG. 13 exemplarily illustrates a G-code, FIG. 14 is a detailed flowchart of a printing process to which the dragging technique is applied, FIG. 15 is a conceptual diagram of the apparatus of manufacturing the artificial esophageal scaffold for performing a printing process to which the dragging technique is applied, FIG. 16 is a picture of the manufactured scaffold, and FIG. 17 is a picture of a strand.

The method of manufacturing the artificial esophageal scaffold having a multi-layered construct using three-dimensional bioprinting according to one embodiment of the present invention may apply the dragging technique to manufacture pores with a size significantly smaller than the nozzle diameter without being constrained by the rotating mandrel, develop a construct that mimics the structure, shape, and cell layer of the esophagus as it is by selectively printing a bioink derived from a natural polymer on each layer, which is collagen, matrigel, fibrin, hyaluronic acid, and so on including esophagus-derived decellularized bioink with cells inside, so it may be possible to develop the artificial esophagus most similar to the function of an actual esophagus.

Referring to FIGS. 3 to 6, the artificial esophageal scaffold (three-layered construct and two-layered construct) manufactured by the method of manufacturing the artificial esophageal scaffold having a multi-layered construct using three-dimensional bioprinting according to one embodiment is shown.

In FIGS. 3a, 4 and 5, the artificial esophageal scaffold 200a of the three-layer construct including an outer layer 210a, a middle layer 220a, and an inner layer 230a is shown, and in FIGS. 3b and 6, the artificial esophageal scaffold 200b of the two-layer construct including an outer layer 210b and an inner layer 230b is shown.

The artificial esophageal scaffold 200a or 200b (hereinafter collectively referred to as ‘200’) of the outer layer 210a or 210b (hereinafter collectively referred to as ‘210’) has a columnar shape (cylinder) having a circular cross section.

In addition, the middle layer 220a and the inner layer 230a or 230b (hereinafter collectively referred to as ‘230’) may be a columnar construct mimicking the mucosa of the esophagus.

For example, the columnar construct may be a wrinkled construct having a columnar shape (hereinafter also referred to as ‘wrinkled column’) having a star-shaped cross-section in which the ridges 242 and the valleys 244 are repeated. The wrinkled column mimics the shape characteristics of the esophagus, which moves food through contraction and relaxation.

Alternatively, the columnar construct may have a columnar shape (cylindrical, elliptical, and so on) having a circular or elliptical cross-section.

Hereinafter, for the convenience of understanding and description of the invention, it is assumed that the columnar construct is the wrinkled construct.

Referring to FIG. 5, very small pores are formed by the dragging technique in the outer layer 210a, the middle layer 220a, and the inner layer 230a to mimic the function of the actual esophagus. The size of the pores may gradually increase from the outer layer 210a to the inner layer 230a.

The inner layer 230, the middle layer 220a (not shown in the two-layer construct), and the outer layer 210 have the same central axis, and diameters may gradually increase from the inner layer 230 to the outer layer 210. Between each layer, an interlayer support 252, 254, 256 (hereinafter collectively referred to as ‘250’) for maintaining the interlayer spacing and maintaining the multi-layered construct of the artificial esophageal scaffold 200 may be disposed. The construct of the artificial esophageal scaffold 200 will be described in detail later.

Referring to FIG. 2, according to the method of manufacturing the artificial esophageal scaffold having the multi-layered construct using three-dimensional bioprinting according to one embodiment, the apparatus of manufacturing the artificial esophageal scaffold first designs the multi-layered construct mimicking the esophagus in S110. The multi-layered esophageal mimicking construct has an elongated cylindrical shape as shown in [1] of FIG. 12, and a hollow columnar shape layer having one or more star-shaped cross-sections may be included therein.

When designing the construct shape, the construct properties such as the diameter, length, and shape of the esophageal mimicking construct can be determined during the design process according to the site to which the artificial esophagus will be applied.

When the design of the construct shape is completed, the dragging technique design is applied to the construct shape in S120.

Hereinafter, the dragging technique will be described with reference to related drawings.

The apparatus of manufacturing the artificial esophageal scaffold according to one embodiment of the present invention may be a three-dimensional printer using a three-dimensional plotting method.

Referring to FIG. 7, the apparatus of manufacturing the artificial esophageal scaffold 1 may include a chamber 10, an air supply unit 20, an injection nozzle 30, and a stage 40.

A polymer material suitable for living tissue (for example, esophagus) is accommodated in the chamber 10. The polymer material may be supplied in a required amount from a separate storage tank (not shown).

The chamber 10 may include a heater (not shown), so that the polymer material accommodated therein can be melted to be easily discharged.

The air supply unit 20 supplies air into the chamber 10 so that the polymer material is discharged through the injection nozzle 30 by pneumatic pressure.

The injection nozzle 30 is installed in the lower part of the chamber 10, and the polymer material accommodated in the chamber 10 is discharged on the stage 40. In this case, the polymer material discharged in the downward direction has a size dependent on an inner diameter of the injection nozzle 30.

The chamber 10 in which the injection nozzle 30 is installed may be configured to be freely movable in the XYZ direction. Alternatively, the stage 40 may be configured to be freely movable in the XYZ direction. Alternatively, the chamber 10 in which the injection nozzle 30 is installed may be configured to be freely movable in n (n is a natural number less than or equal to 2) directions among the XYZ directions, and the stage 40 may be configured to be freely movable in the remaining directions.

Due to the three-dimensional mobility between the chamber 10 in which the injection nozzle 30 is installed and the stage 40, it may be possible to manufacture a three-dimensional construct.

The construct may be designed to have a construct sliced into multiple horizontal layers. The apparatus of manufacturing the construct 1 according to one embodiment cures the polymer material discharged from the chamber 10 through the injection nozzle 30 by pneumatic for each horizontal layer, so that the construct pieces separated by horizontal layers (sliced layers of the construct) are sequentially stacked to form a complete construct.

In this case, according to a plotting technique, the polymer material is discharged in the downward direction through the injection nozzle 30. Accordingly, fibers discharged through the injection nozzle 30 have a size corresponding to the inner diameter of the injection nozzle 30. For example, if the inner diameter of the injection nozzle 30 is 150 μm, the discharged fibers also have a size of about 150 μm.

In one embodiment, in order to overcome this limitation, fibers having a size smaller than the inner diameter of the injection nozzle 30 can be discharged through the dragging technique that controls a discharge parameter in the process of discharging the polymer material through the injection nozzle 30.

FIG. 8 shows a conventional plotting technique for manufacturing construct. It is assumed that a construct having two columns 51a and 51b spaced apart from each other is manufactured.

When the discharging is completed so that the sliced layer is produced by plotting through the injection nozzle 30 with respect to the first column 51a, the injection nozzle 30 is moved in the upward direction. And, after the injection nozzle 30 is moved horizontally to be placed above the second column 51b and then lowered, the plotting for manufacturing the sliced layer corresponding to the second column 51b is performed. In this case, the plotted sliced layer of the first column 51a and the plotted sliced layer of the second column 51b have a size (for example, diameter) corresponding to the inner diameter of the injection nozzle 30.

FIGS. 9 and 10 illustrate the dragging technique for manufacturing the construct according to one embodiment and the construct manufactured by the dragging technique.

Like FIG. 8, after plotting through the injection nozzle 30 for the first column 51a of a construct having two columns 51a and 51b spaced apart by a predetermined interval, rising, horizontally moving, and lowering of the injection nozzle 30 are omitted, and the injection nozzle 30 is directly horizontally moved to the second column 51b.

That is, the dragging method can move the injection nozzle 30 in a direction crossing the discharge direction of the polymer material (i.e., horizontal direction) to make a horizontal bar-structured strand 60 with a reduced diameter by stretching the material being discharged from the injection nozzle 30 or the remaining material in the horizontal direction.

At this time, by controlling the discharge parameter of the molten polymer material through the injection nozzle 30, it becomes possible to form the strand 60 that is horizontally stretched between the plotting on the first column 51a and the plotting on the second column 51b due to the viscosity of the material.

In the drawing, the columnar construct becomes a main frame constituting the construct, and the strand 60 formed between the columns 51a and 51b becomes a sub-frame. In this case, the main frame may be manufactured by the plotting technique, and the sub-frame may be manufactured by the dragging technique between the plotting.

In the dragging technique, the controllable discharge parameter may include a discharge pressure Q, a velocity V, a distance between columns d, a pathway of the injection nozzle 30, an air pressure, and a material viscosity.

The discharge pressure Q is related to a size and length of the strand 60. When the discharge pressure Q is high, it is possible to manufacture the relatively long strand 60 by the dragging technique. In addition, when the discharge pressure Q is low, the size (diameter) of the strand 60 can be made small.

The velocity V is proportional to the size of the strand 60. When the velocity V is fast, the size of the strand 60 becomes small because there is less amount of material descended in the discharge direction at each point on the pathway. When the velocity V is slow, the size of the strand 60 becomes large because there is more amount of material descended in the discharge direction at each point on the pathway.

The distance d between the columns is the minimum length of the strand 60. When the strand 60 made by the dragging technique has a length greater than the distance d between the columns, it may be possible to play a role of creating micropores in the finally manufactured construct.

The pathway of the injection nozzle 30 is related to the arrangement of the strand 60. The thickness of the strand 60 gradually decreases along the pathway of the injection nozzle 30 and may be made horizontally. The strand 60 made by this dragging technique may have a significantly smaller size (for example, diameter) compared to the columns 51a and 51b corresponding to the main frame, so that the strand 60 may partition a plurality of micropores belonging to macro pores formed between the columns in the construct.

In S120, the dragging technique design is applied to the construct shape for which the basic design is completed so that the above-described dragging technique can be applied.

Referring to FIG. 11, a detailed flowchart of the dragging technique design process is shown.

Referring to [1] of FIG. 12, the basic construct designed in S110 is shown.

Based on the shape of the basic design construct, the design is modified to a dragging-applied construct in which a plurality of columns are disposed for each layer as shown in [2] of FIG. 12.

To this end, first, the pore size for each layer is set in S121. Here, each layer refers to the inner layer and the middle layer (omitted in the two-layer construct), both having the wrinkled column shape, and the outer layer of a columnar shape, all disposed in an outward direction from the inside of the artificial esophageal scaffold.

In the artificial esophageal scaffold, the pores of the outer layer may be formed smaller than that of the inner layer. The supply of the cell culture medium is facilitated through the relatively large pores of the inner layer, so that the cells of the corresponding layer can rapidly proliferate. Further, each component of the food flowing through the artificial esophagus in the future may smoothly pass between the layers through the pores of the inner layer.

When the pore sizes for each layer are set, the arrangements of the columns for each layer are set in S122. In one embodiment, the dragging technique is applied when forming pores in the manufacture of each layer of the artificial esophageal scaffold. Therefore, in the dragging technique, the distance between the columns on both sides on which the horizontal strands that form pores are placed acts as an important design factor.

The size of pore can be adjusted by adjusting the distance between the columns in S123 (see [3] of FIG. 12). For example, if the distance between the columns is increased, the length of the horizontal strand becomes longer, and the diameter reduction slope between both ends of the horizontal strand is decreased, so that the size of the pores formed between the horizontal strands disposed vertically may decrease.

Conversely, if the distance between the columns is reduced, the length of the horizontal strand is shortened, and the diameter reduction slope between both ends of the horizontal strand is increased, so that the size of the pores formed between the other horizontal strands disposed vertically may increase.

By taking advantage of these features, by adjusting the distance between the columns on each layer to complete the arrangement of the columns, the design to which the dragging technique is applied can be completed in S124.

In addition, as will be described later in the case of the artificial esophageal scaffold, the columns can be disposed basically at the points corresponding to the ridge and the valley in the star-shaped cross section due to the geometrical features of the wrinkled column shape. In addition, an additional column may be disposed according to the size of the pore to be formed between the column corresponding to the ridge and the column corresponding to the valley.

When the dragging technique design is completed, a G-code to which the dragging technique is applied is generated in S130. G-code is a command code for controlling the operation (move, feed, and so on) of the injection nozzle 30 for the three-dimensional printing.

In one embodiment, the basic design shape in S110, the dragging technique design in S120, and the G-code in S130 may be designed and generated by the control unit (not shown).

Referring to FIG. 13, an example of G-code to which the dragging technique is applied is shown. When seeing the G-code, it is divided into a part of column forming speed, a part of forming column, and a part of nozzle move parameter between the columns. The columns for each layer previously designed in S120 are sequentially formed by the part of column forming speed and the part of forming column. And between the columns, the dragging technique is applied while the injection nozzle 30 is horizontally moved by the part of nozzle move parameter between the columns to form the horizontal strand with a reduced diameter.

When the generation of G-code is completed, the three-dimensional printing to which the dragging technique is applied is performed in S140. A detailed flowchart of the three-dimensional printing process is shown in FIG. 14, and FIG. 15 is a conceptual diagram of a manufacturing apparatus for three-dimensional printing to which the dragging technique is applied.

Referring to FIG. 15, the apparatus of manufacturing the artificial esophageal scaffold 1 supplies air 22 for discharging the molten polymer material through the air supply unit 20. When the polymer material is discharged through the injection nozzle 30 installed in the lower part of the chamber 10, a fan 50 for rapid cooling through forced convection from the outside is installed on the side of the chamber 10 through the fan holder 52. The fan 50 is provided with a fan operation switch 54 on the side so that it can be manually operated. Alternatively, the on/off of the fan 50 may be automatically controlled by a control signal output from the controller (not shown).

Referring to FIG. 14, the G-code to which the dragging technique is applied for controlling the apparatus of manufacturing the artificial esophageal scaffold 1 to perform the three-dimensional printing is input in S141.

Then, the fan 50 is operated to cause forced convection to the injection nozzle 30 in S142, and printing is performed according to the G-code in an environment that cools the injection nozzle 30 in S143.

In this case, the operation or a speed of the fan 50 (On/Off) may be controlled according to each part of the G-code, such as the part of forming column, the part of nozzle move parameter between the columns, and the like. This is because, when the dragging technique is applied, the diameter change of the discharged material can be induced according to the degree to which the discharged material is cooled through forced convection even when the injection nozzle 30 moves at the same velocity.

Through this, in the printing process passing between the column part and the column part quickly, the viscous discharged material is stretched to form the horizontal strand, and it is possible to manufacture a strand having a thickness smaller than the diameter of the injection nozzle 30.

Referring to FIG. 16, the construct manufactured in this way and the pore size formed are shown, and referring to FIG. 17, a strand having a thickness (10 μm or less) smaller than the inner diameter (100 μm) of the injection nozzle is shown.

Parameters in the printing process using the dragging technique may include forced convection through the fan 50, the velocity when moving between the columns, the discharge pressure of the material, the inner diameter of the injection nozzle 30, a feed rate of the injection nozzle 30 and so on.

In addition, the design parameters of the dragging technique may include the distance between the columns, a Z-axis stacking height according to the size of the injection nozzle 30, and the like.

By applying the design parameters of the dragging technique and the printing process parameters together, it is possible to manufacture a construct having a strand smaller than the nozzle inner diameter (for example, about 1/10).

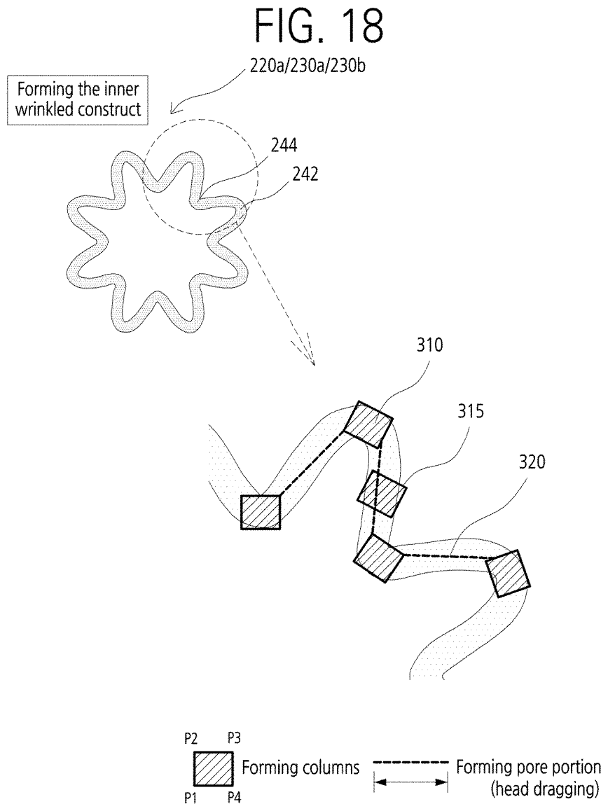

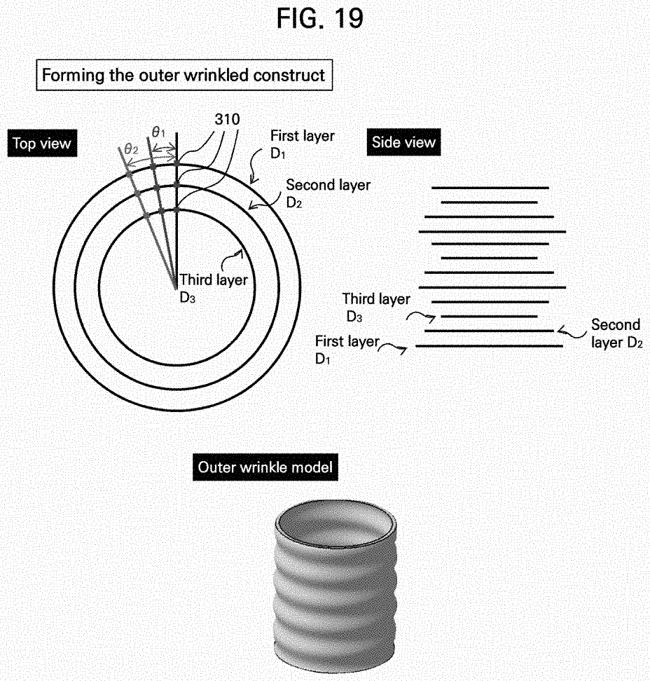

FIG. 18 illustrates the design parameters for forming the inner wrinkles of the artificial esophageal scaffold, and FIG. 19 illustrates the design parameters for forming the outer wrinkles of the artificial esophageal scaffold.

Referring to FIG. 18, the inner layer 230b in the two-layered construct or the inner layer 230a or the middle layer 220a in the three-layered construct is illustrated. These layers are formed in the star-shaped cross section and have a plurality of wrinkles formed in parallel in a lengthwise direction around them by repeating the ridges 242 and valleys 244 extending in the lengthwise direction.

In this case, the columns 310 are disposed by default at positions corresponding to the ridges 242 and the valleys 244.

The column 310 may be formed by four-point injection that sequentially moves the nozzles along the circumference of the column in the order of P1-P2-P3-P4. The column 310 corresponds to the main frame forming a basic skeleton, and allows to secure rigidity by performing four-point injection.

In addition, the pore portion 320 indicated by a dotted line may be formed between the columns 310 by forming a strand of which diameter is gradually reduced by the dragging technique. The length of the pore portion 320 may be determined by the distance between the columns 310. Since the size of the pore changes according to the length of the pore portion 320, the pore size may be determined by the distance between the columns 310.

An auxiliary column 315 may be disposed between the columns 310. The auxiliary column 315 may reduce the distance between the columns 310, thereby reducing the length of the pore portion 320 formed therebetween, and allowing the pore size to be varied accordingly. That is, by disposing the auxiliary column 315, by adjusting the spacing, and so on, pores of a desired size can be formed on the corresponding surface.

The length (i.e., the distance between the columns) of the strand (pore portion) formed by the dragging technique in the artificial esophageal scaffold according to one embodiment is related to the size of the pores, and the size (i.e., thickness) of the column itself formed in the plotting technique is related to a flexibility of the construct. Therefore, the flexibility (or thickness) of the construct can be adjusted by changing the design parameters of P1 to P4.

In addition, in the case of the artificial esophageal scaffold of the three-layered construct, the first and second interlayer supports (252, 254) are disposed between the outer layer (210a) and the middle layer (220a), and between the middle layer (220a) and the inner layer (230a), respectively, it is possible to maintain the spacing between the layers and to have each layer be coaxial. Here, the interlayer supports 252 and 254 may be disposed between the columns of each layer. This is because the column is a part (main frame) that forms a more rigid skeleton than the strand (pore portion).

For stable support, the first interlayer support 252 and/or the second interlayer support 254 may be provided N each, and may be disposed at an interval of 360°/N. For example, if N=4, they may be disposed at intervals of 90°.

Referring to FIG. 19, the outer wrinkles are shown. It will be described with reference to the outer layer 210.

The outer layer 210 is composed of a plurality of sliced horizontal layers (layer 1 to layer N). In this case, by adjusting the diameter of each horizontal layer, the cylindrical outer layer 210 may have a wrinkled construct such as a bellows.

For example, when the first layer has a diameter D1, the second layer has a diameter D2, and the third layer has a diameter D3 (D1>D2>D3), the diameters are gradually reduced. Then, the 4^{th} layer again has the diameter D2 and the 5^{th} layer has the diameter D1, so that the diameters can be gradually increased. By repeating this diameter adjustment for each layer, it is possible to create a bellows-shaped outer wrinkle. That is, the outer wrinkled construct can be formed by repeating the increase and decrease of the diameter according to the height of the horizontal layer.

In this case, the length of the dragging section may be determined according to the distance between the columns, that is, the diameter of each horizontal layer and the angle between the columns of the corresponding horizontal layer.

When the angle between the columns is set to be the same for each horizontal layer, the distance between the columns may vary depending on the diameter of each horizontal layer. That is, the distance between the columns of the third layer may be the shortest, and the distance between the columns of the first layer may be the longest.

Referring back to FIG. 2, when a basic frame printing for the artificial esophageal scaffold 200 is completed through three-dimensional printing, bioink can be printed thereafter in S150. Printing with the bioink may be performed using the three-dimensional printer as shown in FIG. 7. The bioink may be, for example, an esophageal-derived decellularized bioink containing cells from each layer of the esophagus to be printed. Alternatively, it may be a bioink derived from natural polymers such as collagen, matrigel, fibrin, and hyaluronic acid, or an decellularized extracellular matrix-based bioink.

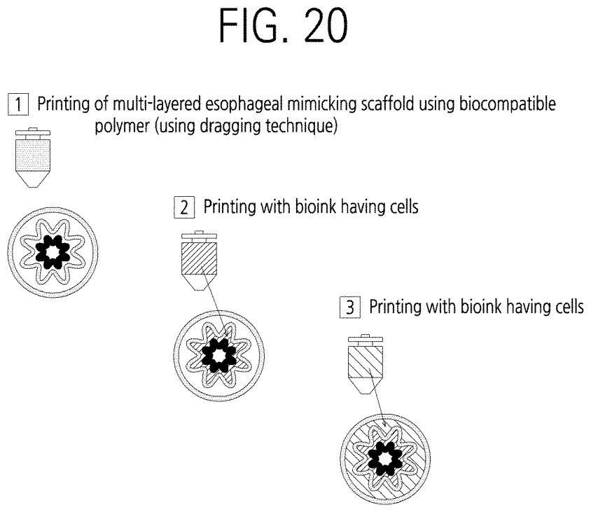

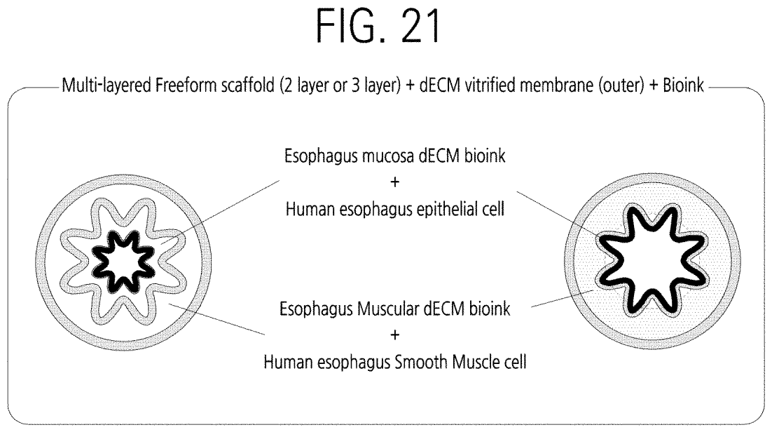

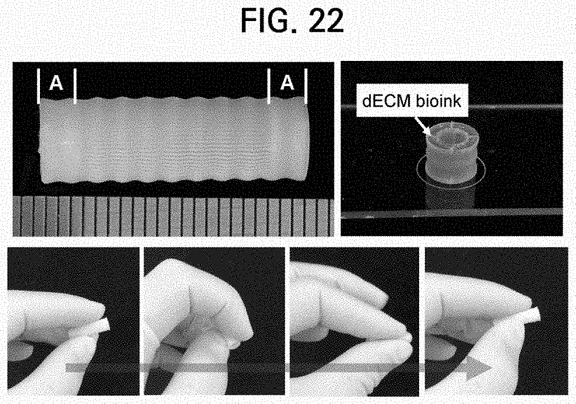

FIG. 20 is a schematic diagram of the artificial esophageal scaffold manufacturing procedure, FIG. 21 illustrates the types of bioink included in the artificial esophageal scaffold.

Referring to FIG. 20, the bioink may be printed in the space between each layer of the three-dimensionally printed artificial esophageal scaffold 200. That is, the same or different bioinks may be printed in the space between the inner layer 230a and the middle layer 220a and the space between the middle layer 220a and the outer layer 210a.

When the printing with the bioink is completed in this way, the manufacture of the artificial esophageal scaffold having a multi-layered construct can be completed in S160.

Referring to FIG. 21, in the case of the artificial esophageal scaffold 200a having the three-layered construct, esophageal mucosa dECM bioink and human esophagus epithelial cells can be injected between the middle layer 220a and the inner layer 230a. In addition, esophageal muscle dECM bioink and human esophagus smooth muscle cells may be injected between the middle layer 220a and the outer layer 210a.

In the case of the artificial esophageal scaffold 200b having the two-layered construct, esophageal mucosa dECM bioink and human esophagus epithelial cells may be coated on the inner surface of the inner layer 230b. In addition, esophageal muscle dECM bioink and human esophagus smooth muscle cells may be injected between the inner layer 230b and the outer layer 210b.

FIG. 22 shows the appearance and flexibility test of the artificial esophageal scaffold manufactured by the method of manufacturing the artificial esophageal scaffold according to one embodiment of the present invention.

Both ends (indicated as A) in the lengthwise direction of the artificial esophageal scaffold 200 are relatively high in rigidity by a certain length, and are manufactured in a structure without pores. This is to ensure excellent retention when the cut portion of the existing esophagus and the structure are stitched during transplantation of the artificial esophageal scaffold 200. In this case, both ends of the artificial esophageal scaffold 200 may be formed only by the plotting technique.

Referring to the lower part of FIG. 22, it can be seen that the flexibility is determined according to the design parameters of the column, can be easily bent by hand, and has a restoring force that is restored to its original state when the external pressure is removed.

According to the embodiment, by applying the dragging technique, the porous construct having the three-dimensional multi-layered construct can be manufactured, and by selectively printing the esophagus-derived bioink containing cells on each layer it can be possible to manufacture the construct mimicking structure, shape and cell layer of the esophagus. Accordingly, it is possible to manufacture the artificial esophagus most similar to the function of the actual esophagus.

In addition, by using the dragging technique, the 4-axis control unit is not required, and it is possible to manufacture a thin porous cylindrical construct and a porous wrinkled columnar construct by stacking in the z-axis. In addition, it is possible to manufacture not only an I-type but also a free-shaped cylindrical construct such as a Y-type.

Although the above has been described with reference to the embodiments of the present invention, those of ordinary skill in the art will understand that various modifications and variations of the present invention can be made without departing from the spirit and scope of the present invention as set forth in the appended claims.

What is claimed is:

1. An artificial esophageal scaffold of a multi-layered construct, comprising:
a first layer in a hollow columnar shape with a circular cross section;
a second layer being disposed inside the first layer and in a columnar construct mimicking a mucosa of an esophagus; and
an interlayer support being disposed between the first layer and the second layer and configured for maintaining the interlayer spacing,
wherein each of the first layer and the second layer comprises a plurality of columns disposed at regular intervals, and a plurality of strands formed by a dragging technique between the plurality of columns,
wherein it has a porous construct by pores between the plurality of strands.

2. The artificial esophageal scaffold of claim 1, wherein the columnar construct has a columnar shape having a star-shaped cross-section,
wherein the columns are disposed at a ridge and a valley of the star-shaped cross-section in the second layer.

3. The artificial esophageal scaffold of claim 2, wherein an auxiliary column is further disposed between the ridge and the valley.

4. The artificial esophageal scaffold of claim 1, wherein the first layer forms an outer wrinkled construct by repeating an increase and a decrease of a diameter according to a height of a sliced horizontal layer.

5. The artificial esophageal scaffold of claim 1, wherein a bioink is printed in a space between the first layer and the second layer.

6. The artificial esophageal scaffold of claim 1, wherein both ends in a lengthwise direction of the artificial esophageal scaffold are manufactured in a structure without pores by a plotting technique.

7. The artificial esophageal scaffold of claim 1, wherein a size of pore is adjusted by a distance between the columns.

8. The artificial esophageal scaffold of claim 1, wherein the columns are formed by a four-point injection plotting technique and are related to a flexibility of the construct.

9. A method of manufacturing an artificial esophageal scaffold of a multi-layered construct, comprising:
separately designing, by a controller in an apparatus of manufacturing the artificial esophageal scaffold, based on a basic design shape for the artificial esophageal scaffold of the multi-layered construct having a first layer in a columnar shape with a circular cross section and a second layer being disposed inside the first layer and in a columnar construct mimicking a mucosa of an esophagus, each layer of the artificial esophageal scaffold into a plurality of columns to be formed by a plotting technique and a strand between the columns to be formed by a dragging technique;
generating G-code to which the dragging technique applied from the separately designed construct; and
manufacturing the columns and the strand according to the G-code,
wherein when the strand is manufactured, a fan of the apparatus of manufacturing the artificial esophageal scaffold is operated to control a degree of stretch of the strand by discharging through the injection nozzle in a state in which forced convection is generated.

10. The method of claim 9, wherein in the separately designing, a size of pore is adjusted by setting pore sizes of each layer and by adjusting a distance between the columns by setting column arrangement at each layer.

11. The method of claim 9, wherein the columnar construct has a columnar shape having a star-shaped cross-section,
wherein in the separately designing, the columns are disposed at a ridge and a valley of the star-shaped cross-section in the second layer.

12. The method of claim 9, wherein in the separately designing, the first layer forms an outer wrinkled construct by repeating an increase and a decrease of a diameter according to a height of a sliced horizontal layer.

13. The method of claim 9 further comprising printing a bioink in a space between the first layer and the second layer.
